(19) United States
(12) Patent Application Publication
(10) Pub. No.: US 2020/0297746 A1
(43) Pub. Date: Sep. 24, 2020

(54) COMPOSITION FOR REDUCING WEIGHT AND LOWERING LIPID, PREPARATION METHOD THEREFOR AND USE THEREOF

(71) Applicant: CATCH BIO-SCIENCE & TECHNOLOGY CO., LTD., ZHANGJIAGANG (CN)

(72) Inventor: YAOLIN WEN, ZHANGJIAGANG (CN)

(73) Assignee: CATCH BIO-SCIENCE & TECHNOLOGY CO., LTD.

(21) Appl. No.: 16/858,588

(22) Filed: Apr. 25, 2020

Related U.S. Application Data
(63) Continuation of application No. PCT/CN2018/073112, filed on Jan. 18, 2018.

(30) Foreign Application Priority Data
Oct. 25, 2017 (CN) 201711012580.6

(51) Int. Cl. A61K 31/7034; A61K 36/9066; A61K 36/48

(52) U.S. Cl. CPC A61K 31/7034; A61K 36/48; A61K 36/9066

(57) ABSTRACT

A composition for reducing weight and lowering lipid, a preparation method therefor, and a use thereof. The pharmaceutical composition comprises the following crude drugs: 18-65 parts by weight of phloridzin or pharmaceutically acceptable salts thereof; and 15-78 parts by weight of a turmeric extract. The pharmaceutical composition has the effects of weight reducing and lipid lowering, and the pharmaceutical composition has better effects of weight reducing and lipid lowering than a single crude drug.

### CROSS-REFERENCE TO RELATED APPLICATIONS

This application is a continuation of International Patent Application No. PCT/CN2018/073112 with a filing date of Jan. 18, 2018, designating the United States, now pending, and further claims priority to Chinese Patent Application No. 201711012580.6 with a filing date of Oct. 25, 2017. The content of the aforementioned applications, including any intervening amendments thereto, are incorporated herein by reference.

### TECHNICAL FIELD

The invention belongs to the field of health-care products or medicines, and relates to a pharmaceutical composition for reducing weight and lowering lipid and a preparation method therefor and a use thereof.

### BACKGROUND OF THE INVENTION

Obesity is the most common chronic endocrine metabolic disease, the incidence of which is increasing year by year. As a result, obesity has become a global public health problem. The degree of obesity is indicated generally by the Body Mass Index (BMI). According to the International Standard formulated in a meeting of experts of the World Health Organization (WHO), a BMI value in the range of 24≤BMI<28 is considered overweight, and a BMI value of over 30 is considered obese. Statistical results of the WHO show that more than one billion adults were overweight over the world and of these at least 300 million were obese. The number of overweight people will reach 1.5 billion in 2015 if no effective measures are taken. Obesity can cause many health problems, not only can increase the incidence and mortality of hypertension, coronary heart disease, type 2 diabetes, and can also cause respiratory complications, osteoarthritis and mental diseases.

Currently, methods of weight loss mainly comprise reducing weight by medicine or by diet. The current weight-reducing medicine mainly comprises the following two broad categories: pancreatic lipase inhibitors and appetite suppressants. Pancreatic lipase inhibitors can inhibit the activity of the pancreatic lipase, thereby further inhibiting the decomposition and absorption of fat in the food, thus realizing weight loss. Appetite suppressants are restricted for use because they may cause adverse reactions of the nervous system. To date, the FDA approved weight loss drugs that can be used for a long term only include Orlistat which is a lipase inhibitor, Lorcaserin which is a 5-Hydroxytryptamine 2C receptor agonist, and Qsymia which is a weight reducing compound (a sustained release agent comprising phenylbutylamine and topiramate). However, these weight-reducing drugs can cause adverse reactions such as fat diarrhea, fat-soluble vitamin deficiency and the like, and have great uncertainty on toxic or side effects on the brain hub and the cardiovascular system or the like.

Hyperlipemia is a systemic disease which involves higher levels of one or more lipids in plasma than normal levels due to abnormal fat metabolism or transport. The main features of hyperlipemia include increased levels of triacylglycerol (TG), total cholesterol (TC) and low density lipoprotein cholesterol (LDL-C) and reduced level of high density lipoprotein cholesterol (HDL-C) in serum. Hyperlipemia is one of the causes of cardiovascular and cerebrovascular diseases. According to statistics, about 12 million people died from cardiovascular and cerebrovascular diseases worldwide every year. At present, clinical first-line drugs for reducing blood fat are mainly statins which have exact and fast curative effects, but have the disadvantages of easy rebound, high recurrence rate and long-term medicine taking. In 2001, cerivastatin was withdrawn from market because of fatal rhabdomyolysis in a subject, a severe muscle adverse reaction, which elicits concerns and heated discussions on the safety of other statins drugs.

Although in the prior art it is reported that phloridzin has a blood lipid lowering effect and an extract from Curcuma longa L. can significantly reduce the blood lipid level of rats and rabbits, there have been no report that a pharmaceutical composition consisting of phloridzin and an extract from Curcuma longa L. has effects of reducing blood lipid or losing weight.

### SUMMARY OF THE INVENTION

In a first aspect, in some embodiments the present invention provides a pharmaceutical composition comprising the following crude drugs:

18 to 65 parts by weight of phloridzin or a pharmaceutically acceptable salt thereof, and 15 to 78 parts by weight of an extract from Curcuma longa L., based on the weight of phloridzin.

Preferably, the pharmaceutical composition comprises the following crude drugs: 22 to 65 parts by weight of phloridzin or a pharmaceutically acceptable salt thereof; and 35 to 78 parts by weight of an extract from Curcuma longa L., based on the weight of phloridzin.

Preferably, the pharmaceutical composition comprises the following crude drugs:

65 parts by weight of phloridzin and 35 parts by weight of an extract from Curcuma longa L., or

22 parts by weight of phloridzin and 78 parts by weight of an extract from Curcuma longa L., or

45 parts by weight of phloridzin and 55 parts by weight of an extract from Curcuma longa L.

Preferably, the extract from Curcuma longa L. is prepared with a method comprising the following steps: collecting rhizomes of Curcuma longa L., extracting the rhizomes with alcohols, merging the resulted extraction solutions, followed by concentrating. More preferably, the extract from Curcuma longa L. is prepared with a method comprising the following steps: collecting rhizomes of Curcuma longa L., extracting the rhizomes by heating under reflux for 1 to 5 times, each time extracting for 0.5 to 3 hours with an aqueous solution of ethanol having a volume concentration of 10-50% in an amount of 5 to 10 times the weight of the rhizomes, merging the resulted extraction solutions, followed by concentrating and drying.

More preferably, the extract from Curcuma longa L. is prepared with a method comprising the following steps: collecting rhizomes of Curcuma longa L., extracting the rhizomes by heating under reflux for 2 times, each time extracting for 1 hour with an aqueous solution of ethanol having a volume concentration of 30% in an amount of 8 times the weight of the rhizomes, merging the resulted extraction solutions, followed by concentrating and drying.

Preferably, the pharmaceutical composition comprises the following crude drugs:

21 to 45 parts by weight of epigallocatechin gallate or a pharmaceutically acceptable salt thereof, based on the weight of epigallocatechin gallate.

More preferably, the pharmaceutical composition comprises the following crude drugs: 19 to 60 parts by weight of phloridzin or a pharmaceutically acceptable salt thereof; and 15 to 36 parts by weight of an extract from Curcuma longa L., based on the weight of phloridzin; and 25 to 45 parts by weight of epigallocatechin gallate or a pharmaceutically acceptable salt thereof, based on the weight of epigallocatechin gallate.

More preferably, the pharmaceutical composition comprises the following crude drugs:

60 parts by weight of phloridzin, 15 parts by weight of an extract from Curcuma longa L., and 25 parts by weight of epigallocatechin gallate, or

19 parts by weight of phloridzin, 36 parts by weight of an extract from Curcuma longa L., and 45 parts by weight of epigallocatechin gallate, or

35 parts by weight of phloridzin, 32 parts by weight of an extract from Curcuma longa L., and 33 parts by weight of epigallocatechin gallate.

Preferably, the above pharmaceutical composition further comprises 20 to 59 parts by weight of an extract from sennae folium.

More preferably, the pharmaceutical composition comprises the following crude drugs: 18 to 37 parts by weight of phloridzin or a pharmaceutically acceptable salt thereof, 23 to 30 parts by weight of an extract from Curcuma longa L., and 33 to 59 parts by weight of an extract from sennae folium, based on the weight of phloridzin.

More preferably, the pharmaceutical composition comprises the following crude drugs:

18 parts by weight of phloridzin, 23 parts by weight of an extract from Curcuma longa L., and 59 parts by weight of an extract from sennae folium, or

37 parts by weight of phloridzin, 30 parts by weight of an extract from Curcuma longa L., and 33 parts by weight of an extract from sennae folium, or

22 parts by weight of phloridzin, 27 parts by weight of an extract from Curcuma longa L., and 51 parts by weight of an extract from sennae folium.

Preferably, the extract from sennae folium is prepared with a method comprising the following steps: collecting sennae folium, extracting the sennae folium with alcohols, merging the resulted extraction solutions, followed by concentrating.

Preferably, the extract from sennae folium is prepared with a method comprising the followings steps: collecting sennae folium, extracting the sennae folium by heating under reflux for 1 to 5 times, each time extracting for 0.5 to 3 hours with an aqueous solution of ethanol having a volume concentration of 10-30% in an amount of 8 to 12 times the weight of the sennae folium, merging the resulted extraction solutions, followed by concentrating and drying.

More preferably, the extract from sennae folium is prepared with a method comprising the followings steps: collecting sennae folium, extracting the sennae folium by heating under reflux for 2 times, each time extracting for 1 hour with an aqueous solution of ethanol having a volume concentration of 15% in an amount of 10 times the weight of the sennae folium, merging the resulted extraction solutions, followed by concentrating and drying.

Preferably, the pharmaceutical composition further comprises the following crude drugs: 21 to 45 parts by weight of epigallocatechin gallate or a pharmaceutically acceptable salt thereof, and 20 to 59 parts by weight of an extract from sennae folium, based on the weight of epigallocatechin gallate.

In some embodiments, the present invention provides a pharmaceutical composition, comprising the following crude drugs: 20 to 24 parts by weight of phloridzin or a pharmaceutically acceptable salt thereof; and 33 to 41 parts by weight of an extract from Curcuma longa L., based on the weight of phloridzin, and 19 to 23 parts by weight of epigallocatechin gallate or a pharmaceutically acceptable salt thereof, and 18 to 22 parts by weight of an extract from sennae folium, based on the weight of epigallocatechin gallate.

Preferably, the above pharmaceutical composition comprises the following crude drugs:

22 parts by weight of phloridzin, 37 parts by weight of an extract from Curcuma longa L., 21 parts by weight of epigallocatechin gallate, and 20 parts by weight of an extract from sennae folium, or

20 parts by weight of phloridzin, 41 parts by weight of an extract from Curcuma longa L., 19 parts by weight of epigallocatechin gallate, and 22 parts by weight of an extract from sennae folium, or

24 parts by weight of phloridzin, 33 parts by weight of an extract from Curcuma longa L., 23 parts by weight of epigallocatechin gallate, and 18 parts by weight of an extract from sennae folium,

In a second aspect, in some embodiments the present invention further provides a preparation method of the pharmaceutical composition, comprising the following steps:

weighing out selected parts by weight of phloridzin or a pharmaceutically acceptable salt thereof and the extract from Curcuma longa L., and mixing them well by grinding; or

weighing out selected parts by weight of phloridzin or a pharmaceutically acceptable salt thereof, the extract from Curcuma longa L. and epigallocatechin gallate or a pharmaceutically acceptable salt thereof, and mixing them well by grinding; or weighing out selected parts by weight of phloridzin or a pharmaceutically acceptable salt thereof, the extract from Curcuma longa L. and the extract from sennae folium, and mixing them well by grinding; or

weighing out selected parts by weight of phloridzin or a pharmaceutically acceptable salt thereof, the extract from Curcuma longa L., epigallocatechin gallate or a pharmaceutically acceptable salt thereof and the extract from sennae folium, and mixing them well by grinding.

In a third aspect, in some embodiments the present invention provides a pharmaceutical formulation, comprising the pharmaceutical composition as described above as an active ingredient, mixed with a conventional auxiliary material and prepared according to a conventional process into a clinically acceptable dosage form selected from the group consisting of tablets, capsules, powders, pills, granules, syrups, injections, solutions, mixtures, lotions, liniments, membranes, plasters, ointments, suppositories, pastes, gelatins, aerosols and sprays.

The conventional auxiliary material is selected from the group consisting of a filler, a disintegrating agent, a lubricant, a suspending agent, an adhesive, a sweetener, a flavoring agent, a preservative, a matrix and the like. The filler is selected from the group consisting of starch, pregelatinized starch, lactose, mannitol, chitin, microcrystalline cellulose, sucrose, and the like. The disintegrating agent is selected from the group consisting of starch, pregelatinized starch, microcrystalline cellulose, sodium carboxymethyl starch, cross-linked polyvinylpyrrolidone, low substituted hydroxypropyl cellulose, croscarmellose sodium, and the like. The lubricant is selected from the group consisting of magnesium stearate, sodium dodecyl sulfate, talcum powder, silicon dioxide and the like. The suspending agent is selected from the group consisting of polyvinylpyrrolidone, microcrystalline cellulose, sucrose, agar, hydroxypropyl methyl cellulose and the like. The adhesive is selected from the group consisting of starch slurry, polyvinylpyrrolidone, hydroxypropyl methyl cellulose and the like. The sweetener is selected from the group consisting of sodium saccharin, aspartame, sucrose, sodium cyclamate, glycyrrhizic acid and the like. The flavoring agent is selected from the group consisting of a sweetener and various essences. The preservative is selected from the group consisting of paraben, benzoic acid, sodium benzoate, sorbic acid and salts thereof, benzalkonium bromide, chlorhexidine acetate, Eucalyptus oil and the like. The matrix is selected from the group consisting of PEG 6000, PEG 4000, insect wax and the like.

In a fourth aspect, in some embodiments the present invention provides a use of the pharmaceutical composition or the pharmaceutical formulation as described above in the manufacture of a medicament or health care product for reducing weight.

In a fifth aspect, in some embodiments the present invention provides a use of the pharmaceutical composition or the pharmaceutical formulation as described above in the manufacture of a medicament or health care product for lowering lipid.

In a sixth aspect, the present invention provides a medicament or health care product for reducing weight or lowering lipid, comprising the pharmaceutical composition as described above.

In a seventh aspect, the present invention provides a method of reducing weight or lowering lipid, comprising administering the pharmaceutical composition as described above or administering the pharmaceutical formulation as described above, or

administering the medicament or health care product as described above to a subject in need.

In an eighth aspect, the present invention provides a method of preparing a medicament or health care product for reducing weight or lowering lipid, comprising using the pharmaceutical composition or the pharmaceutical formulation as described above as a raw material.

The technical solutions of the present invention have the following advantages:

(1) In a research of the present invention, it is found that the pharmaceutical composition prepared from specific parts by weight of phloridzin or a pharmaceutically acceptable salt thereof and specific parts by weight of an extract from Curcuma longa L. has a remarkable weight-reducing effect which is much better than the weight-reducing effect of a single crude drug, showing a synergistic effect in weight-reducing. Moreover, the pharmaceutical composition has a remarkable blood lipid lowering effect which is much better than the blood lipid lowering effect of a single crude drug, also showing a synergistic effect in blood lipid lowering.

(2) In a further research of the present invention, it is found that the pharmaceutical composition prepared from specific parts by weight of phloridzin or a pharmaceutically acceptable salt thereof, specific parts by weight of an extract from Curcuma longa L. and specific parts by weight of epigallocatechin gallate or a pharmaceutically acceptable salt thereof has a remarkable weight-reducing effect which is significantly better than the weight-reducing effect of a single crude drug, showing a synergistic effect in weight-reducing; Moreover, the pharmaceutical composition has a remarkable blood lipid lowering effect which is significantly better than the blood lipid lowering effect of a single crude drug, also showing a synergistic effect in blood lipid lowering.

(3) In a further research of the present invention, it is found that the pharmaceutical composition prepared from specific parts by weight of phloridzin or a pharmaceutically acceptable salt thereof, specific parts by weight of an extract from Curcuma longa L. and specific parts by weight of an extract from sennae folium has a remarkable weight-reducing effect which is significantly better than that of a single crude drug, showing a synergistic effect in weight-reducing; Moreover, the pharmaceutical composition has a remarkable blood lipid lowering effect which is significantly better than the blood lipid lowering effect of a single crude drug, showing a synergistic effect in blood lipid lowering.

(4) In a further research of the present invention, it is found that the medicinal composition prepared from specific parts by weight of phloridzin or a pharmaceutically acceptable salt thereof, specific parts by weight of an extract from Curcuma longa L., specific parts by weight of epigallocatechin gallate or a pharmaceutically acceptable salt thereof and specific parts by weight of an extract from sennae folium has a weight-reducing effect which is significantly better than the weight-reducing effect of a single crude drug, showing a synergistic effect in weight-reducing; Moreover, the pharmaceutical composition has a remarkable blood lipid lowering effect which is significantly better than the blood lipid lowering effect of a single crude drug, showing a synergistic effect in blood lipid lowering.

(5) In a further research of the present invention, it is found that the extract from Curcuma longa L. is prepared by extraction with an aqueous solution of ethanol having a specific concentration of 10-50%, so that active ingredients in Curcuma longa L. can be extracted to the greatest extent, and thus the pharmaceutical composition prepared from such obtained extract from Curcuma longa L. combined with other crude drugs has more remarkable effects of weight reducing and blood lipid lowering.

(6) In a further research of the present invention, it is found that the extract from sennae folium is prepared by extraction with an aqueous solution of ethanol having a specific concentration of 10-30%, so that active ingredients in sennae folium can be extracted to the greatest extent, and thus the pharmaceutical composition prepared from such obtained extract from sennae folium combined with other crude drugs has more remarkable effects of weight reducing and blood lipid lowering.

### DETAILED DESCRIPTION

Materials used in the following examples and experiment examples of the invention:

(1) Phloridzin, which is commercially available and has a purity of more than or equal to 98%.

(2) An extract from Curcuma longa L., which is prepared according to the following steps: collecting and pulverizing dried rhizomes of Curcuma longa L., extracting the rhizomes by heating under reflux for 2 times, each time extracting for 1 hour with an aqueous solution of ethanol having a volume concentration of 30% in an amount of 8 times the weight of the rhizomes, merging the resulted extraction solutions, followed by concentrating and drying.

(3) An extract from sennae folium, which is prepared according to the following steps: collecting and pulverizing dried sennae folium, extracting the sennae folium by heating under reflux for 2 times, each time extracting for 1 hour with an aqueous solution of ethanol having a volume concentration of 15% in an amount of 10 times the weight of the sennae folium, merging the resulted extraction solutions, followed by concentrating and drying.

### Example 1

Provided is a pharmaceutical composition, comprising the following crude drugs: 65 g of phloridzin and 35 g of the extract from Curcuma longa L.

The pharmaceutical composition is prepared with a method comprising the following steps: respectively weighing out the above selected weight of phloridzin and the extract from Curcuma longa L., and mixing them well by grinding.

The pharmaceutical composition is mixed with a conventional auxiliary material and prepared into tablets according to a conventional process.

### Example 2

Provided is a pharmaceutical composition, comprising the following crude drugs: 22 g of phloridzin and 78 g of the extract from Curcuma longa L.

The pharmaceutical composition is prepared with a method comprising the following steps: respectively weighing out the above selected weight of phloridzin and the extract from Curcuma longa L., and mixing them well by grinding.

The pharmaceutical composition is mixed with a conventional auxiliary material and prepared into capsules according to a conventional process.

### Example 3

Provided is a pharmaceutical composition, comprising the following crude drugs: 45 g of phloridzin and 55 g of the extract from Curcuma longa L.

The pharmaceutical composition is prepared with a method comprising the following steps: respectively weighing out the above selected weight of phloridzin and the extract from Curcuma longa L., and mixing them well by grinding.

The pharmaceutical composition is mixed with a conventional auxiliary material and prepared into granules according to a conventional process.

### Example 4

Provided is a pharmaceutical composition, comprising the following crude drugs: 60 g of phloridzin, 15 g of the extract from Curcuma longa L., and 25 g of epigallocatechin gallate.

The pharmaceutical composition is prepared with a method comprising the following steps: respectively weighing out the above selected weight of phloridzin, the extract from Curcuma longa L. and epigallocatechin gallate, and mixing them well by grinding.

The pharmaceutical composition is mixed with a conventional auxiliary material and prepared into tablets according to a conventional process.

### Example 5

Provided is a pharmaceutical composition, comprising the following crude drugs: 19 g of phloridzin, 36 g of the extract from Curcuma longa L., and 45 g of epigallocatechin gallate.

The pharmaceutical composition is prepared with a method comprising the following steps: respectively weighing out the above selected weight of phloridzin, the extract from Curcuma longa L. and epigallocatechin gallate, and mixing them well by grinding.

The pharmaceutical composition is mixed with a conventional auxiliary material and prepared into granules according to a conventional process.

### Example 6

Provided is a pharmaceutical composition, comprising the following crude drugs: 18 g of phloridzin, 23 g of the extract from Curcuma longa L., and 59 g of the extract from sennae folium.

The pharmaceutical composition is prepared with a method comprising the following steps: respectively weighing out the above selected weight of phloridzin, the extract from Curcuma longa L. and the extract from sennae folium, and mixing them well by grinding.

The pharmaceutical composition is mixed with a conventional auxiliary material and prepared into capsules according to a conventional process.

### Example 7

Provided is a pharmaceutical composition, comprising the following crude drugs: 37 g of phloridzin, 30 g of the extract from Curcuma longa L., and 33 g of the extract from sennae folium.

The pharmaceutical composition is prepared with a method comprising the following steps: respectively weighing out the above selected weight of phloridzin, the extract from Curcuma longa L. and the extract from sennae folium, and mixing them well by grinding.

The pharmaceutical composition is mixed with a conventional auxiliary material and prepared into tablets according to a conventional process.

### Example 8

Provided is a pharmaceutical composition, comprising the following crude drugs: 22 g of phloridzin, 37 g of the extract from Curcuma longa L., 21 g of epigallocatechin gallate, and 20 g of the extract from sennae folium.

The pharmaceutical composition is prepared with a method comprising the following steps: respectively weighing out the above selected weight of phloridzin, the extract from Curcuma longa L., epigallocatechin gallate, and the extract from sennae folium, and mixing them well by grinding.

The pharmaceutical composition is mixed with a conventional auxiliary material and prepared into tablets according to a conventional process.

### Experiment Example 1: Study of Weight Reducing and Lipid Lowering Effect of Compositions of the Invention

### 1. Experimental Materials

Cholesterol and sodium cholate are purchased from Shanghai Great Wall Pharmaceutical Co., Ltd. (Shanghai, China).

The feed is commercially available. The high-fat feed comprises 75% base feed, 2% cholesterol, 0.5% sodium cholate, 15% lard, and 7.5% egg yolk. 150 male Spague-Dawlay (SD) Rats of clean grade, each aged 4 weeks and weighed 150-180 g, are provided by Shanghai Lingchang Biotechnology Co., Ltd. The rats are fed in separate plastic cages, given free access to water and feed for 7 days to adapt to the environment, and then quarantined.

### 2. Experimental Methods

2.1 Grouping

140 rats of similar weight are selected from the 150 rats, randomly divided into 14 groups, with 10 rats in each group, including experimental groups 1-8, comparative experiment groups 1-4, a model control group and a blank control group. Rats in the blank control group are fed with the base feed, and rats in other groups are fed with the high-fat feed.

2.2 Administration Methods

Rats in the experimental groups 1-8 are respectively administered via lavage with 80 mg/kg of the pharmaceutical compositions prepared in Examples 1-8; rats in the comparative experiment groups 1-4 are respectively administered via lavage with 80 mg/kg of phloridzin, 80 mg/kg of the extract from Curcuma longa L., 80 mg/kg of epigallocatechin gallate and 80 mg/kg of the extract from sennae folium; and rats in the model control group and the blank control group are administered via lavage with equivalent amounts of saline.

Rats in each group receive administration once daily for 7 continuous weeks.

### 3. Testing and Processing of Experimental Data

3.1 Testing Index

(1) After 7 weeks of administration, the weight and weight gain of rats in each group are observed and recorded;

(2) Rats are anesthetized with ether, and fat pads surrounding the genitalia and kidney and the fat of the omentum are separated and weighed. A fat-to-weight ratio is calculated according to the following equation:

fat-to-weight ratio (%)=(weight of fat surrounding the genitalia+weight of fat surrounding kidney+weight of fat of the omentum)/body weight×100%;

(3) After 7 weeks of administration, blood are collected from orbital posterior venous plexus of the rats and analyzed with an enzymatic method on a full-automatic biochemical analyzer to quantitatively determine levels of triglycerides (TG), total cholesterol (TC), high density lipoprotein cholesterol (HDL-C), and low density lipoprotein cholesterol (LDL-C) in serum.

3.2 Statistical Analysis

Data processing is performed using SPSS 20.0 software. Differences between the groups are analyzed using a single-factor variance analysis.

### 4. Experimental Results

4.1 Weight Reducing Results

After 7 weeks of administration, the weight gain and fat-to-weight ratio results of rats of each group are shown in Table 1.

TABLE 1
Body weight gain and fat-to-weight ratio of rats of each
group (x̅ ± s, n = 10)

| | Body weight | Fat-to-weight |
|---|---|---|
| Groups | gain (g) | ratio (%) |
| Blank Control Group | 131.52 ± 22.75 | 3.68 ± 0.38 |
| Model Control Group | 197.11 ± 17.43^{##} | 7.82 ± 0.87^{##} |
| Experimental Group 1 | 141.55 ± 19.98** | 4.21 ± 0.42** |
| Experimental Group 2 | 142.37 ± 20.06** | 4.09 ± 0.39** |
| Experimental Group 3 | 145.33 ± 20.67** | 4.23 ± 0.52** |
| Experimental Group 4 | 149.47 ± 18.96** | 4.29 ± 0.36** |
| Experimental Group 5 | 142.51 ± 19.51** | 4.03 ± 0.44** |
| Experimental Group 6 | 148.89 ± 20.04** | 4.35 ± 0.46** |
| Experimental Group 7 | 147.39 ± 18.28** | 4.30 ± 0.52** |
| Experimental Group 8 | 152.33 ± 20.37** | 5.27 ± 0.55* |
| Comparative experiment | 161.35 ± 20.01* | 5.87 ± 0.72* |
| Group 1 | | |
| Comparative experiment | 175.19 ± 24.41 | 7.18 ± 0.76 |
| Group 2 | | |
| Comparative experiment | 189.76 ± 22.25 | 7.48 ± 0.53 |
| Group 3 | | |
| Comparative experiment | 186.33 ± 19.59 | 7.37 ± 0.49 |
| Group 4 | | |

Note:
Compared with the blank control group,
P^{##} < 0.01, and compared with the model control group,
P** < 0.01, and
P* < 0.05.

Table 1 indicates that:

(1) After 7 weeks of administration, rats in the model control group exhibit very significant body weight gain and fat-to-weight (P<0.01) when compared with the blank control group, indicating obesity model is successfully established;

(2) Compared with the model control group, the weight gain of the rats in the experimental groups 1-8 is significantly reduced (P<0.01), which indicates that the pharmaceutical compositions prepared in Examples 1-8 can significantly reduce weight and slow weight gain;

(3) Compared with the model control group, the rats in the comparative experiment groups 1-4 have a tendency to reduce body weight, but the body weight reducing effect is not as significant as the body weight reducing effect in experimental groups 1-8.

4.2 Blood Lipid Lowering Results

After 7 weeks of administration, experimental results of the blood biochemical parameters of rats of each group are shown in Table 2.

TABLE 2
Blood biochemical parameters of rats of each group (x̅ ± s, n = 10)

| | TG | TC | HDL-C | LDL-C |
|---|---|---|---|---|
| Groups | (mmol/L) | (mmol/L) | (mmol/L) | (mmol/L) |
| Blank Control Group | 1.21 ± 0.39 | 1.78 ± 0.36 | 0.69 ± 0.11 | 0.29 ± 0.08 |
| Model Control Group | 1.98 ± 0.46^{##} | 2.65 ± 0.47^{##} | 0.58 ± 0.15 | 0.57 ± 0.11^{##} |
| Experimental Group 1 | 1.43 ± 0.37* | 2.11 ± 0.35* | 0.71 ± 0.12 | 0.41 ± 0.11* |
| Experimental Group 2 | 1.39 ± 0.31* | 2.08 ± 0.40* | 0.68 ± 0.14 | 0.37 ± 0.10** |
| Experimental Group 3 | 1.41 ± 0.29* | 1.95 ± 0.41** | 0.72 ± 0.13 | 0.33 ± 0.09** |
| Experimental Group 4 | 1.38 ± 0.41* | 1.91 ± 0.33** | 0.84 ± 0.17 | 0.42 ± 0.06* |
| Experimental Group 5 | 1.29 ± 0.38** | 2.12 ± 0.49* | 0.70 ± 0.08 | 0.37 ± 0.08** |
| Experimental Group 6 | 1.44 ± 0.44* | 1.92 ± 0.53** | 0.68 ± 0.14 | 0.41 ± 0.09* |
| Experimental Group 7 | 1.50 ± 0.51* | 2.02 ± 0.45* | 0.77 ± 0.09 | 0.43 ± 0.12* |
| Experimental Group 8 | 1.49 ± 0.48* | 2.05 ± 0.51* | 0.71 ± 0.16 | 0.40 ± 0.07* |
| Comparative experiment | 1.56 ± 0.39* | 2.21 ± 0.51 | 0.72 ± 0.10 | 0.49 ± 0.10 |
| Group 1 | | | | |
| Comparative experiment | 1.61 ± 0.24 | 2.45 ± 0.50 | 0.79 ± 0.07 | 0.58 ± 0.09 |
| Group 2 | | | | |
| Comparative experiment | 1.77 ± 0.41 | 2.55 ± 0.43 | 0.59 ± 0.12 | 0.55 ± 0.12 |
| Group 3 | | | | |
| Comparative experiment | 1.82 ± 0.46 | 2.58 ± 0.49 | 0.62 ± 0.11 | 0.52 ± 0.08 |
| Group 4 | | | | |

Note:
Compared with the blank control group, P^{##} < 0.01, and compared with the model control group,
P** < 0.01, and
P* < 0.05.

Table 2 indicates that:

(1) After 7 weeks of administration, rats in the model control group exhibit very significant increase (P<0.01) in the blood biochemical parameters of TG, TC and LDL-C when compared with the blank control group, indicating hyperlipemia model is successfully established;

(2) Compared with the model control group, the reduction in the blood biochemical parameters of TG, TC, LDL-C of rats in the experimental groups 1-8 is significant or very significant (P<0.05 or P<0.01);

(3) Compared with the model control group, the reduction in the blood biochemical parameters of TG, TC, LDL-C of rats in the comparative experiment groups 1-4 is not significant.

### 5. Experimental Conclusions

(1) The pharmaceutical composition of the present invention has a remarkable weight-reducing effect which is significantly better than that of a single crude drug, showing a synergistic effect; and (2) the pharmaceutical composition of the present invention has a balanced reduction effect on the levels of TG, TC and LDL-C of the hyperlipemia rats, and the blood lipid reducing effect of the pharmaceutical composition is significantly better than that of a single crude drug, showing a synergistic effect.

It is to be understood that the above-described embodiments are merely illustrative of the embodiments and are not intended to be limiting of the embodiments. It will be apparent to one of ordinary skill in the art that other different forms of changes or variations can be made on the basis of the above description. The embodiments need not be exhaustive. It is to be understood that various changes or modifications may be made herein without departing from the scope of the invention as defined by the appended claims.

1. A pharmaceutical composition, comprising the following crude drugs:
18 to 65 parts by weight of phloridzin or a pharmaceutically acceptable salt thereof, and 15 to 78 parts by weight of an extract from Curcuma longa L., based on the weight of phloridzin.

2. The pharmaceutical composition of claim 1, comprising the following crude drugs:
22 to 65 parts by weight of phloridzin or a pharmaceutically acceptable salt thereof; and 35 to 78 parts by weight of an extract from Curcuma longa L., based on the weight of phloridzin.

3. The pharmaceutical composition of claim 1 or 2, further comprising the following crude drugs:
21 to 45 parts by weight of epigallocatechin gallate or a pharmaceutically acceptable salt thereof, based on the weight of epigallocatechin gallate.

4. The pharmaceutical composition of claim 3, comprising the following crude drugs:
19 to 60 parts by weight of phloridzin or a pharmaceutically acceptable salt thereof; and 15 to 36 parts by weight of an extract from Curcuma longa L., based on the weight of phloridzin; and
25 to 45 parts by weight of epigallocatechin gallate or a pharmaceutically acceptable salt thereof, based on the weight of epigallocatechin gallate.

5. The pharmaceutical composition of claim 1 or 2, further comprising the following crude drugs: 20 to 59 parts by weight of an extract from sennae folium.

6. The pharmaceutical composition of claim 5, comprising the following crude drugs:
18 to 37 parts by weight of phloridzin or a pharmaceutically acceptable salt thereof, 23 to 30 parts by weight of an extract from Curcuma longa L., and 33 to 59 parts by weight of an extract from sennae folium, based on the weight of phloridzin.

7. The pharmaceutical composition of claim 1, further comprising the following crude drugs:
21 to 45 parts by weight of epigallocatechin gallate or a pharmaceutically acceptable salt thereof, and 20 to 59 parts by weight of an extract from sennae folium, based on the weight of epigallocatechin gallate.

8. The pharmaceutical composition of claim 7, comprising the following crude drugs:
20 to 24 parts by weight of phloridzin or a pharmaceutically acceptable salt thereof; and 33 to 41 parts by weight of an extract from Curcuma longa L., based on the weight of phloridzin, and
19 to 23 parts by weight of epigallocatechin gallate or a pharmaceutically acceptable salt thereof, and 18 to 22 parts by weight of an extract from sennae folium, based on the weight of epigallocatechin gallate.

9. The pharmaceutical composition of claim 1, wherein the pharmaceutical composition is used as an active ingredient and mixed with a conventional auxiliary material and prepared according to a conventional process into a pharmaceutical formulation in a clinically acceptable dosage form selected from the group consisting of tablets, capsules, powders, pills, granules, syrups, injections, solutions, mixtures, lotions, liniments, membranes, plasters, ointments, suppositories, pastes, gelatins, aerosols and sprays.

10. A medicament or health care product for reducing weight or lowering lipid, comprising the pharmaceutical composition of claim 1.

11. A method of reducing weight or lowering lipid, comprising administering the pharmaceutical composition of claim 1 to a subject in need.

12. The method of claim 11, wherein the pharmaceutical composition comprises the following crude drugs:
22 to 65 parts by weight of phloridzin or a pharmaceutically acceptable salt thereof; and 35 to 78 parts by weight of an extract from Curcuma longa L., based on the weight of phloridzin.

13. The method of claim 11, wherein the pharmaceutical composition further comprises the following crude drugs:
21 to 45 parts by weight of epigallocatechin gallate or a pharmaceutically acceptable salt thereof, based on the weight of epigallocatechin gallate.

14. The method of claim 13, wherein the pharmaceutical composition comprises the following crude drugs: 19 to 60 parts by weight of phloridzin or a pharmaceutically acceptable salt thereof; and 15 to 36 parts by weight of an extract from Curcuma longa L., based on the weight of phloridzin; and
25 to 45 parts by weight of epigallocatechin gallate or a pharmaceutically acceptable salt thereof, based on the weight of epigallocatechin gallate.

15. The method of claim 11, wherein the pharmaceutical composition further comprises the following crude drugs: 20 to 59 parts by weight of an extract from sennae folium.

16. The method of claim 15, wherein the pharmaceutical composition comprises the following crude drugs: 18 to 37 parts by weight of phloridzin or a pharmaceutically acceptable salt thereof, 23 to 30 parts by weight of an extract from Curcuma longa L., and 33 to 59 parts by weight of an extract from sennae folium, based on the weight of phloridzin.

17. The method of claim 11, wherein the pharmaceutical composition further comprises the following crude drugs:
21 to 45 parts by weight of epigallocatechin gallate or a pharmaceutically acceptable salt thereof, and 20 to 59 parts by weight of an extract from sennae folium, based on the weight of epigallocatechin gallate.

18. The method of claim 17, wherein the pharmaceutical composition comprises the following crude drugs: 20 to 24 parts by weight of phloridzin or a pharmaceutically acceptable salt thereof; and 33 to 41 parts by weight of an extract from Curcuma longa L., based on the weight of phloridzin, and
19 to 23 parts by weight of epigallocatechin gallate or a pharmaceutically acceptable salt thereof, and 18 to 22 parts by weight of an extract from sennae folium, based on the weight of epigallocatechin gallate.

19. The method of claim 11, wherein the pharmaceutical composition is administered at a dosage of 80 mg/kg.

20. The method of claim 19, wherein the pharmaceutical composition is administered once daily for 7 continuous weeks.
